(19) United States
(12) Patent Application Publication
(10) Pub. No.: US 2014/0057296 A1
(43) Pub. Date: Feb. 27, 2014

(54) ASSAYS FOR ANTIBODIES WHICH BIND TO THERAPEUTIC AGENTS

(71) Applicants: Juan Ruiz, Acton, MA (US); Marcia Sellos-Moura, West Newbury, MA (US)

(72) Inventors: Juan Ruiz, Acton, MA (US); Marcia Sellos-Moura, West Newbury, MA (US)

(73) Assignee: Shire Human Genetic Therapies, Inc., Lexington, MA (US)

(21) Appl. No.: 14/002,567

(22) PCT Filed: Mar. 1, 2012
(86) PCT No.: PCT/US12/27329

§ 371 Date: Nov. 12, 2013

Related U.S. Application Data
(60) Provisional application No. 61/448,099, filed on Mar. 1, 2011.

(51) Int. Cl. G01N 33/68

(52) U.S. Cl. CPC G01N 33/6854
USPC 435/7.92

(57) ABSTRACT

Methods and kits for determining the presence in a patient biological sample of an antibody which binds to an agent (e.g., an enzyme administered in the course of ERT), and optionally determining the neutralizing effect or lack thereof of the antibody on the agent are disclosed.

### RELATED APPLICATION(S)

This application claims priority to U.S. Provisional Application No. 61/448,099 filed on Mar. 1, 2011, the entire teachings of which are incorporated herein by reference.

### FIELD OF THE INVENTION

The present inventions are directed to assays, kits and related methods for determining the presence of neutralizing antibodies in a biological sample.

### BACKGROUND OF THE INVENTION

Therapeutic biological agents have become a potent weapon in the treatment of a variety of conditions and disorders. For example, enzyme replacement therapy (ERT) is now available for many of the lysosomal storage diseases (LSDs), including Gaucher disease, Fabry disease, Pompe disease, mucopolysaccharidosis (MPS) type I (Hurler, Scheie and Hurler-Scheie syndromes), MPS II (Hunter syndrome) and MPS VI (Maroteaux-Lamy syndrome). Unfortunately, but not unexpectedly, undesired immunogenicity of administered biological agents is a significant problem in patients to whom these agents are administered, and a subset of patients treated with such agents will typically develop antibodies against the administered therapeutic. These antibodies have the potential to alter the therapeutic efficacy of the agent, with effects ranging from no apparent adverse effect to significant adverse reactions and impaired clinical response to treatment. The identification of patients in whom an unwanted immunogenic response is elicited is important, for example, in establishing clinical courses of treatment and corresponding patient monitoring.

Additionally, the development of antibodies consequent to ERT and the presence of such antibodies in a biological sample may materially interfere with the accurate pharmacokinetic analysis of biological samples (e.g., by causing an inaccurate detection of administered agent in patient serum). The antibodies may interfere with certain ELISA assays used to detect agents administered during the course of ERT due to the antibodies blocking the binding of a capture antibody to the agent (e.g., on assay microtiter plates). The sensitivity of other assays for detecting interfering antibodies, such as indirect ELISA assays used to detect antibodies to agents such as the enzyme idursulfase, may be limited by absorbance of such enzyme's conformational epitopes to the polystyrene surface of the microtiter plate used to conduct the assay, precluding binding and detection of some antibodies.

Needed are sensitive assays and methods for accurately identifying interfering antibodies in a biological sample. Particularly needed are assays and methods to detect the presence of conformation specific antibodies in a sample.

### SUMMARY OF THE INVENTION

Disclosed herein are methods and kits for determining or identifying the presence of an antibody which binds to an agent (e.g., an enzyme administered in the course of ERT) in a patient biological sample, and optionally determining the interfering effect or lack thereof of the antibody on the agent. In certain embodiments, the methods, kits and assays disclosed herein provide accurate and sensitive means of evaluating the pharmacokinetic properties of an agent in a biological sample despite the presence of interfering antibodies in such sample.

For example, disclosed herein are assays and methods (termed “conformation specific assays” or “CSA”) of determining the presence of a target antibody in a biological sample of a patient which binds an agent administered to the patient comprising a step of obtaining the biological sample from a patient to whom an agent of interest has previously been administered; contacting the biological sample with a predetermined amount of the agent of interest under conditions appropriate for binding of the agent to a target antibody in the biological sample, wherein if the target antibody is present an agent-target antibody pair is produced; subsequently contacting the biological sample with a capture agent capable of binding to the agent of interest to produce a captured agent of interest; removing unbound biological sample components; contacting the captured agent of interest with a signaling agent capable of binding to the captured agent of interest; removing unbound signaling agent; and comparing the presence of bound signaling agent to a control, thereby determining the presence or absence of the target antibody in the biological sample.

In some embodiments, the capture agent is immobilized (e.g., a plurality of capture agents may be immobilized, fixed or otherwise bound to the surface of one or more wells of a microtiter plate). In some embodiments the control comprises captured agent of interest that was not previously exposed to the target antibody. In some embodiments the capture agent is a competitive inhibitor of the target antibody for binding to the agent of interest. In some aspects the capture agent binds to the same physical epitope on the agent of interest as is bound by the target antibody, and in some aspects the capture agent binds to the same conformational epitope on the agent of interest as is bound by the target antibody.

In some embodiments, removal of unbound biological sample components comprises a washing step.

In some embodiments, the methods may further comprise comparing the amount of bound signaling agent to a control, thereby determining the amount of target antibody in the biological sample.

In some embodiments, the agent of interest is a therapeutic agent such as an enzyme (e.g., idursulfase).

In some embodiments, the plurality of immobilized capture agents are immobilized on a solid planar surface. In some embodiments the plurality of immobilized capture agents are a plurality of immobilized antibodies.

In some embodiments, the signaling agent is a labeled agent (e.g., a light-emitting label, HRP, etc.). In some embodiments, the signaling agent is a labeled antibody. In certain embodiments, removal of unbound signaling agent comprises a washing step.

In some embodiments, the control is a standard derived from the amount of bound signaling agent in the absence of target antibody in the biological sample. In some embodiments the control is a standard curve plotting amount of bound signaling agent in the presence of varying amounts of target antibody in the biological sample.

In some disclosed embodiments, if the presence of target antibody is detected in the biological sample, a confirmatory assay is optionally performed. If the presence of one or more target antibodies are detected in the biological sample (e.g., patient serum) as a result of assays described herein, such target antibodies (e.g., contained in or isolated from the serum) can be further tested for neutralizing properties with regard to the agent of interest.

In some embodiments, the biological sample comprises serum.

In some embodiments, the biological sample comprises cerebrospinal fluid (CSF).

In one exemplary, non-limiting example, a blood sample is assayed for target antibody by CSA. Samples that are negative for target antibody can be further screened for target antibody by enzyme-linked immunosorbant assays (ELISA). Samples positive for target antibody as determined by CSA or ELISA can be confirmed using a radioimmunoprecipitation (RIP) assay; if antibody presence is confirmed, target antibody isotype and/or target antibody titer can be determined. In addition, if antibody presence is confirmed, samples can be tested for neutralizing activity using, for example an in vitro enzyme inhibition assay. In certain embodiments the results of the assay(s) are compared with the patient baseline, and results can be reported as percent inhibition of time point compared to baseline. In some embodiments samples which inhibit iduronate-2-sulfatase (I2S) activity by more than 10%, more than 15%, more than 20%, more than 25%, more than 30%, more than 35%, more than 40%, etc., are classified as positive for target antibody having a neutralizing effect with regard to the agent.

The above discussed and many other features and attendant advantages of the present invention will become better understood by reference to the following detailed description of the invention when taken in conjunction with the accompanying examples. The various embodiments described herein are complimentary and can be combined or used together in a manner understood by the skilled person in view of the teachings contained herein.

### BRIEF DESCRIPTION OF THE DRAWINGS

FIG. 1 shows Kaplan-Meier analysis of time from start of ERT to initial infusion-related reaction event in patients who started enzyme replacement therapy at or after enrollment in the Hunter Outcome Survey (HOS) (n=104). Data were censored at 1 year from HOS entry or at death.

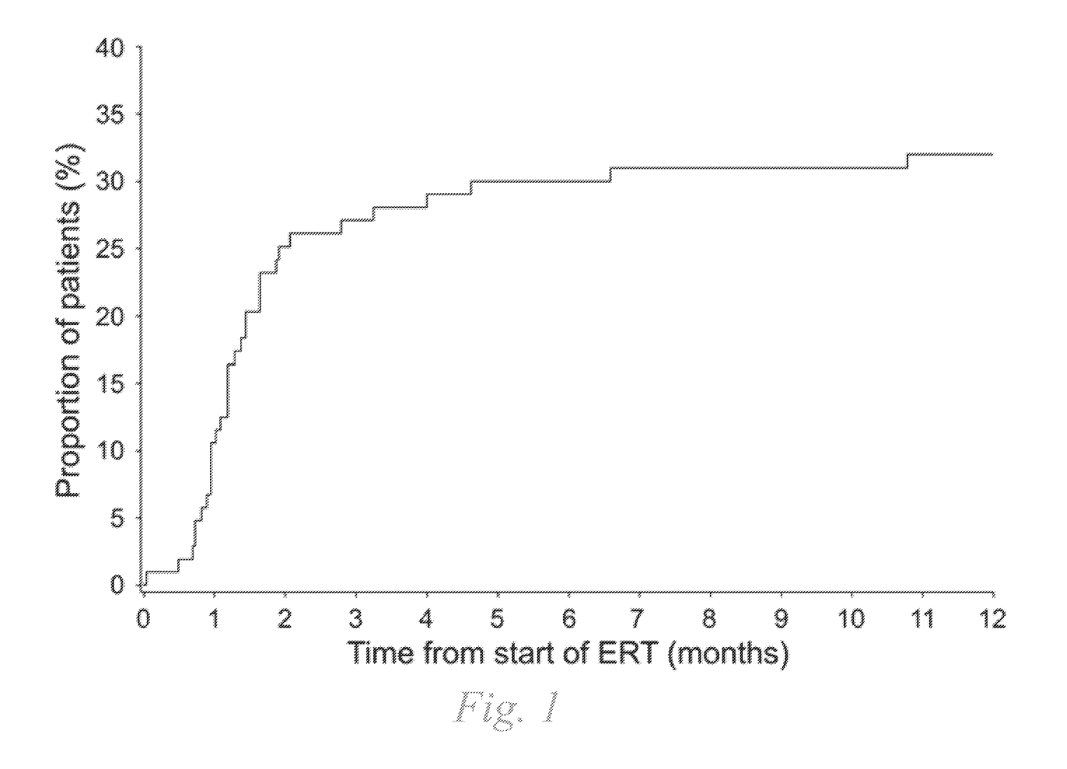

FIG. 2 is a flow chart showing management of infusion-related reaction events to idursulfase. ^{a} IRR is defined as an event occurring during or within 24 hours of an infusion and with evidence of a causal relationship to idursulfase. ^{b} In cases where the patient has a history of mild, readily managed IRR events, the physician may choose to slow the rate of infusion instead of stopping the infusion completely. ^{c} Corticosteroids may be given orally in a single dose on the day prior to infusion or intravenously 1 hour prior to infusion. IRR refers to infusion-related reaction; IV refers to intravenous.

FIG. 3 is an idursulfase standard curve (0-20 ng/mL) prepared in connection with the studies described herein. The curve was analyzed by a 4-parameter logistic model, and the R² value is 1.000.

FIG. 4 is a mean idursulfase standard curve prepared in connection with the validation studies described herein using twenty-two idursulfase standard curves (0-20 ng/mL). The curve was analyzed by a linear regression model, and the R² value (0.991) for the mean idursulfase standard curve demonstrates the assay linearity.

### DETAILED DESCRIPTION OF THE INVENTION

Enzyme replacement therapy (ERT) is now available for many of the lysosomal storage diseases (LSDs), including Gaucher disease, Fabry disease, Pompe disease, mucopolysaccharidosis (MPS) type I (Hurler, Scheie and Hurler-Scheie syndromes), MPS II (Hunter syndrome) and MPS VI (Maroteaux-Lamy syndrome). It is essential that the safety of these therapies be monitored in parallel with assessments of their efficacy. ERT for LSDs has been found to be generally well tolerated and, for most products, the most common adverse events reported in the clinical trials were related to the underlying disease. The most frequent drug-related adverse events were infusion-related reactions (IRRs) (i.e., any events occurring within 24 hours of the infusion considered possibly or probably related to treatment by the treating physician). Signs and symptoms of an IRR can include cutaneous reactions, pyrexia, headache and hypertension.

The majority of IRRs in patients receiving ERT for LSDs are hypersensitivity reactions of the type formerly known as anaphylactoid reactions, with occasional instances of severe allergic reactions. IRRs can occur upon or immediately following the first administration of the drug and are usually dose-dependent; signs and symptoms are generally apparent within 5-60 minutes of exposure to the drug and can typically be reversed by stopping the infusion. However, in some cases, IRRs may not develop until several hours after the infusion or may be biphasic reactions (e.g., signs and symptoms recur after an interval of apparent recovery).

IRRs may have a humoral component and, as is expected following infusion of any protein-based therapy, formation of antibodies to the therapeutic agent has been reported in patients receiving ERT for LSDs (reviewed in Brooks et al., Trends Mol. Med. 9 (2003) 450-453; and Richards, Clinical and Applied Immunology Reviews 2 (2002) 241-253). However, the precise relationship between antibody formation and occurrence of IRRs remains unclear; not all IRRs are antibody-mediated and not all patients who develop antibodies will experience an IRR. Antibodies formed against infused enzymes are usually of the immunoglobulin (Ig) G serotype, but there are rare reports of IgE antibodies in patients receiving some forms of ERT, including agalsidase beta for Fabry disease, laronidase for MPS I, algucosidase alfa for Pompe disease and alglucerase for Gaucher disease. Antibodies formed against infused enzymes or proteins may potentially impact (e.g., reduce) the efficacy of ERT.

Home therapy is increasingly being considered and implemented for patients with LSDs such as Hunter syndrome. For this reason, it is important to understand the timing and incidence of IRRs and establish the best means of managing these events. Accordingly, methods of identifying antibodies (e.g., neutralizing antibodies) formed against administered biological or agents (e.g., enzymes administered as part of an ERT) are useful in this context, among others.

MPS II is a progressive X-linked LSD resulting from a deficiency of the enzyme iduronate-2-sulfatase (I2S). As a consequence of the enzyme deficiency, glycosaminoglycans (GAGs) accumulate over time in lysosomes in tissues throughout the body, leading to progressive multisystem involvement. Patients with a more severe phenotype have central nervous system involvement with progressive neurologic deterioration, and historical reports indicate that they generally do not survive beyond the second decade of life. Patients with a more attenuated form of the disorder exhibit little or no cognitive impairment and may survive well into adult life, although somatic features of the disorder produce considerable morbidity.

ERT with recombinant iduronate-2-sulfatase (idursulfase; e.g., Elaprase®, Shire HGT Inc., Cambridge, Mass., USA) has been available in the United States since 2006 and in Europe since 2007. It is administered weekly by intravenous infusion and has been found to be well tolerated and to result in improvements in many of the signs and symptoms of Hunter syndrome. The most frequent adverse events in the initial clinical trials were those consistent with the clinical manifestations of Hunter syndrome. IRRs are the most commonly reported idursulfase-associated adverse events. Analysis of antibody formation revealed the presence of IgG antibodies to idursulfase in approximately half of the patients in each trial; IgM antibodies were detected in two of these patients. IgE antibodies were not detected in any patient, and no instances of anaphylactic shock have been reported. The presence of antibodies to idursulfase was associated with a transient increase in levels of urinary GAGs, but no effect on clinical efficacy was observed.

As described herein, methods and kits are provided for determining or identifying the presence of an antibody in patient serum which binds to an agent (e.g., an enzyme administered in the course of ERT), and optionally determining the neutralizing effect or lack thereof of the antibody on the agent. Conformation specific antibodies in serum samples form antigen-antibody complexes during a sample pre-incubation step, resulting in a corresponding decrease in the amount of free agent (e.g., idursulfase) detectable based on the formation of antigen-antibody complexes in solution and measurement of such complexes by, for example, competitive immunometric enzyme-linked immunosorbent assays (ELISA).

Antibodies which bind to an agent of interest as described herein, are herein termed “target antibodies.” Target antibodies, if present in a biological sample (e.g., a blood or serum sample) to be tested, result from an immunogenic response in a patient to whom an agent of interest (e.g., an enzyme in a ERT regimen) has been administered. Target antibodies may be of any isotype, including IgG (including IgG1, IgG2, IgG3, and IgG4 subtypes), IgA (including IgA1 and IgA2 subtypes), IgM, and IgE, depending on the immune response mounted by the patient.

An agent of interest can include virtually any agent administered to an individual (e.g., a patient) but is preferably a biological agent (e.g., a recombinantly-prepared enzyme). The methods and kits described herein are particularly well suited for use with enzyme agents, such as enzymes administered in the course of ERT. Suitable enzyme agents include, but are not limited to, lysosomal enzymes. Lysosomal enzymes are required to break down protein, glycolipid and carbohydrate metabolites within the lysosomes of cells. For example, lysosomal enzyme agents of interest in the methods described herein include, but are not limited to, iduronate-2-sulfatase, beta-glucocerebrosidase, alpha-galactosidase A, acid alpha-glucosidase, and beta-N-acetyl-hexosaminidase.

As described herein, the disclosed methods comprise a number of steps. These steps can be performed in any order unless otherwise indicated either explicitly or implicitly (e.g., if the result of a prior step is a necessary predicate to a subsequent step, it is implicit that the order of these two steps may not be reversed).

One step of the disclosed methods comprises obtaining a blood sample from a patient to whom an agent of interest has previously been administered. The blood sample can be separated into one or more fractions, such as a serum fraction, and the serum fraction can be used for subsequent analysis if desired. In some cases a blood sample can be obtained from the same patient prior to administration of the agent of interest, and this sample can serve as a baseline or control.

The sample (e.g., serum sample) is then pre-incubated with a predetermined amount of the agent of interest under conditions appropriate for binding of the agent to a target antibody in the sample (e.g., serum), wherein if the target antibody is present in the sample an agent-target antibody pair or complex is produced. Suitable conditions are known in the art, and certain exemplary conditions are described herein and in the examples below. Alternative suitable conditions will be readily apparent to the skilled artisan. Formation of such agent-target antibody pairs results in a corresponding decrease in the amount of free agent (e.g., idursulfase) which can be detected in subsequent steps of the assay and methods disclosed herein.

The pre-incubated serum is contacted with a capture agent capable of binding to the agent of interest to produce a captured agent of interest. In some embodiments, the capture agent is an antibody (polyclonal or monoclonal, human, goat, etc.) or binding fragment thereof which binds (e.g., specifically) to the agent of interest. For example, the capture agent can be a competitive inhibitor of the target antibody for binding to the agent of interest. In one example, the capture agent binds to the same physical epitope on the agent of interest as is bound by the target antibody; in another example the capture agent binds to the same conformational epitope on the agent of interest as is bound by the target antibody.

In some embodiments the capture agent is immobilized. For example, the capture agent can be immobilized on a solid surface such as a plate or bead or in a microtiter plate well. Suitable surfaces are known in the art. Upon contact with the pre-incubated serum, the capture agent binds to the free agent of interest (e.g., free idursulfase) but does not bind, or binds with significantly lower affinity, to agent contained in an agent-target antibody pair. This binding produces an amount of captured agent of interest which can subsequently be measured directly or indirectly.

The methods and assays disclosed herein can further comprise a step of removing unbound serum components, for example, through use of a wash step standard in the art. Such a wash step is conducted under conditions which remove unbound serum components (e.g., to reduce sample complexity and background noise) but which allow immobilized capture agent to remain immobilized and bound to agent of interest.

A number of methods can be used to measure captured agent of interest. In one embodiment, the captured agent of interest is contacted with a signaling agent capable of binding to the captured agent of interest (e.g., a labeled signaling agent such as a labeled antibody). The signaling agent can be labeled radioactively, luminescently, chemiluminescently, enzymatically, etc. using methods well known in the art. For example, the signaling agent can be labeled with a light-emitting label. In one example the signaling agent can be labeled with horseradish peroxidase (HRP).

Unbound signaling agent can be removed by washing, and the remaining sample subjected to conditions suitable for detection of the signal emitted by the remaining signaling agent. Suitable conditions will be determined based on the signaling moiety used and will be readily apparent to the skilled artisan. For example, if horseradish peroxidase (HRP) is utilized as the label/signaling moiety, the sample can be incubated with the peroxidase substrate 3,3′,5,5′-tetramethyl benzidine (TMB) and the absorbance at 450 nm measured. The amount of bound signaling agent can be compared against a calibration curve for the activity of the agent of interest (e.g., idursulfase), to determine the amount of captured agent of interest. Results can be reported, for example, as the ratio of the result from a patient test sample to that of the patient baseline sample.

The presence or absence of target antibody in the sample can optionally be confirmed using additional assays known in the art, including but not limited to, ELISA assays and RIP assays. Examples of such confirmatory assays are described in the examples herein. Moreover, positive samples can be additionally tested for neutralizing activity by one or more in vitro enzyme inhibition assays when the agent of interest is an enzyme. The conditions of the in vitro enzyme inhibition assay will depend on the identity of the enzyme and will be known to the skilled artisan. Exemplary assays for idursulfase enzyme activity are described herein and in the examples, but it will be understood that the invention is not limited to idursulfase as the agent of interest or to the particulars of the exemplified enzyme inhibition assay.

The results of the methods described herein can be used to guide treatment decisions and clinical monitoring. For example, patients in whom neutralizing antibodies are raised may be monitored more closely for adverse effects and for efficacy of treatment. Such patients may also be candidates for immunosuppressive therapy to minimize the occurrence and/or effect of undesirable immunogenic response to the therapeutic agent.

Also encompassed herein are kits and reagents for performing the disclosed methods. Some or all of the reagents for the disclosed methods can be packaged together along with instructions for use in performing the described assays. By way of non-limiting example, such a kit may comprise some or all of: a free agent of interest, a substrate comprising one or more immobilized capture agents, one or more signaling agents, and one or more agents suitable for detecting said signaling agents. The kit may further comprise additional reagents for performing confirmatory assays and/or in vitro enzyme inhibition assays.

Currently there is only limited information available on the occurrence of IRRs in the “real world” setting for patients (e.g., patients with Hunter syndrome receiving enzyme replacement therapy (ERT)). The Hunter Outcome Survey (HOS) is a global multicenter, long term, observational database designed to collect data from patients with Hunter syndrome in their normal medical care environment. (See, Burton, et al., Mol. Genet. Metab. 103 (2011) 113-120, the entire disclosure of which is incorporated herein by reference.) As part of the work described herein, information in HOS regarding the frequency, timing and severity of reported IRR events during the first year of treatment with idursulfase was analyzed, and formation of antibodies to idursulfase was characterized. The analysis was restricted to patients who started treatment with idursulfase at or after enrollment in HOS and for whom at least 1 year of follow-up data was available (n=104).

A total of 65 infusion related reactions (IRR) events were reported in 33 (31.7%) patients in the first year of enzyme replacement therapy (ERT). Six of these patients experienced more than three events. Nearly all of the initial IRR events occurred during the first 3 months of ERT; five patients (4.8% of the total patient population) experienced their first IRR event after 3 months of treatment. Only two patients (1.9% of the total patient population) experienced their first IRR event after more than 6 months of ERT. Most of the IRR events were of mild-to-moderate severity. After initially stopping the infusion, IRR events were generally readily managed by slowing the infusion and/or use of antihistamines or antipyretics. No patient in this analysis discontinued ERT because of an IRR event. IgG antibodies to idursulfase were detected in 32/63 patients (50.8%) for whom samples were taken; no patient developed IgE to idursulfase. Serum antibody levels were measured within 24 hours of an IRR event for 10 IRR events in 7 patients; 7/9 samples contained IgG to idursulfase, 2 of which had neutralizing activity.

IRR events in patients receiving idursulfase can typically be readily managed without interruption of treatment. Initial IRR events usually occur in the first 3 months of treatment, but in rare instances may occur after more than 6 months of therapy. Physicians using ERT to treat patients with MPS II, either in the clinic or at home, should therefore be familiar with the timing, nature and recommended management of IRR events.

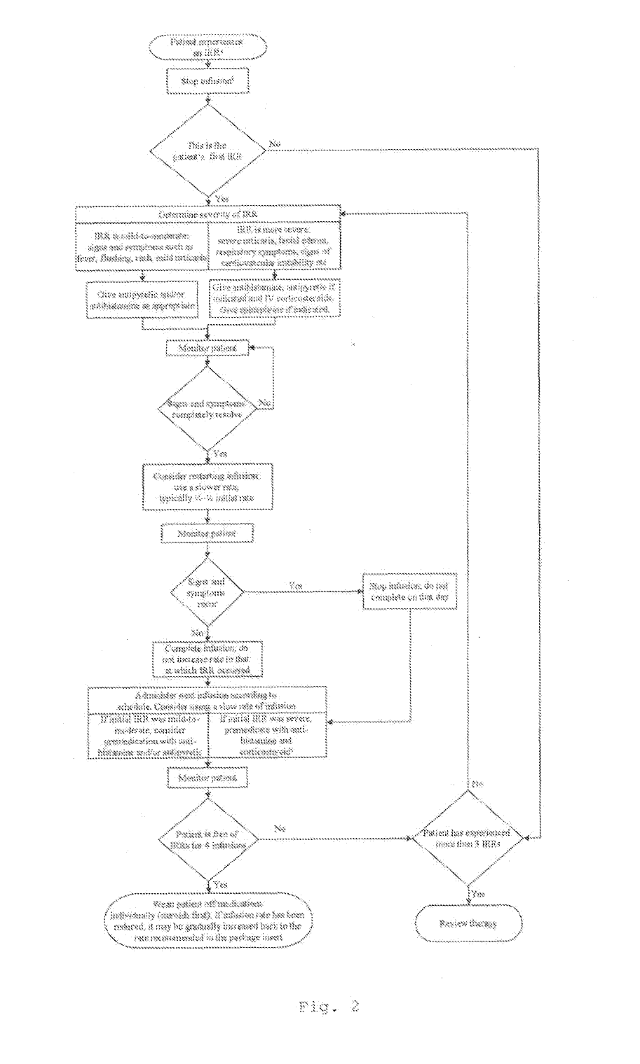

Analysis of data from patients in HOS who had not previously received idursulfase revealed that almost one-third experienced at least one IRR event within the first year of starting ERT. A small but significant number experienced repeated (more than three) IRR events. Most of these IRR events were of mild-to-moderate severity and, after initially halting the infusion, were easily managed by using a slower rate of infusion and/or the use of antihistamines or antipyretics. No patient in this analysis discontinued idursulfase therapy because of an IRR event. Due to the frequency with which IRR events occur, it is clear that physicians using ERT to treat patients with MPS II should be familiar with the timing and nature of IRR events and should be prepared to treat them when they take place. Some guidelines for management of IRR events in patients receiving idursulfase are presented as an algorithm in FIG. 2. In a non-limiting example, as shown in FIG. 2, if the initial reaction is mild or moderate, premedication with antihistamine or antipyretic may be employed. (See, Muenzer et al., Pediatrics 123 (2009) 229-240.) As biphasic reactions have been reported in patients receiving idursulfase, it should be noted that careful observation after any IRR event is recommended.

Consistent with previous reports, approximately half of the patients for whom information was available in this analysis developed IgG antibodies to idursulfase, and there were no instances of IgE formation. The timing and severity of IRR events are key factors when considering whether to recommend transition to home infusions. An important observation described herein was that initial IRR events generally occurred during the first 3 months of treatment. IRR events rarely occurred after 3 months of treatment with ERT and even more rarely after 6 months of treatment. Therefore, sometime between 3 and 6 months after initiation of treatment in a physician-supervised setting (hospital or infusion center) would appear to be a reasonable time to consider a transition to home infusions for patients in whom it is considered appropriate. Individuals administering infusions in the home setting or elsewhere, after this time, should be aware that IRR events may still occur, albeit rarely, and should be prepared to respond appropriately. The assays, methods and kits disclosed herein provide means of objectively monitoring patients receiving ERT and in some instances may serve to predict those patients that are likely to develop IRRs in response to ERT. Similarly, such assays, methods and kits may provide a useful tool to identify those patients that may be less prone to developing IRRs and for whom home infusion ERT may be appropriate.

While certain compounds, compositions and methods of the present invention have been described with specificity in accordance with certain embodiments, the following examples serve only to illustrate the compounds of the invention and are not intended to limit the same. Each of the publications, reference materials, GenBank accession numbers and the like referenced herein to describe the background of the invention and to provide additional detail regarding its practice are hereby incorporated by reference in their entirety.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification.

### EXAMPLES

### Example 1

The present example describes the conformation specific anti-idursulfase (CSA) assay that was used to screen for conformation specific antibodies against idursulfase that may be present in human serum samples. The CSA is based upon the formation of antigen-antibody complexes in solution and measurement of such complexes by competitive immunometric ELISA technology.

### Reagents and Materials

Idursulfase protein: Purified idursulfase (idursulfase), 2.1 mg/mL. For standard curve, use 2 μg/mL idursulfase in dilution buffer (PBS/2% BSA/0.05% Tween-20);
Idursulfase dilution buffer: 8 ng/mL idursulfase (1×) or 16 ng/mL idursulfase (2×) in dilution buffer;
Positive serum for conformation specific anti-idursulfase antibodies;
Negative serum for conformation specific anti-idursulfase antibodies (Bioreclamation Inc., Cat. No. #HMSRM-F);
Background levels of conformation specific anti-idursulfase antibodies: serum samples from patients with Hunter syndrome prior to receiving idursulfase enzyme replacement therapy;
1:400 Positive Control Serum: pooled positive human sera (equal volume mixture of 15 positive serum samples from 11 individuals) 1:400 diluted in dilution buffer;
Goat anti-idursulfase IgG 6.4 mg/mL;
Horseradish peroxidase (HRP)-conjugated goat anti-idursulfase IgG;
TMB Peroxidase EIA Substrate Kit (Pierce Cat. No. 34021);
Immuno Microwell 96-well flat-bottomed Nunc MaxiSorp ELISA plate (Nunc Cat. No. 442404);
Molecular Devices SPECTRAMAX plate reader with attached computer equipped with SOFTMAX PRO software and assay template;
Calibrated 37±0.5° C. shaking plate incubator: JITTERBUG (Boekel Scientific), Bench-Top Incubated Shaker (Barnstead); and
Bio-TEK Automatic Plate Washer (Model No. ELX50).

### Methods

Briefly, the controls and human serum samples were pre-incubated at room temperature for a minimum of two hours with the idursulfase agent without shaking to allow the formation of antigen (idursulfase)-antibody complexes. Standards were prepared from the 2 μg/mL idursulfase stock solution serially diluted as described in Table 1 below. At the same time, microtiter plates were coated with a polyclonal goat anti-idursulfase antibody (IgG) and incubated at 37° C. for one hour, washed and then blocked with dilution buffer for an additional hour. All incubation steps in the microtiter plate were performed at 37° C. with gentle shaking. After blocking, the plates were washed again, and the standards and the pre-incubated samples and controls were added into duplicate wells of the microtiter plate and incubated at 37° C. for one hour. The microtiter plates were then washed to remove unbound materials and subsequently incubated with a horseradish peroxidase (HRP)-conjugated goat anti-idursulfase detection antibody (IgG) for one hour at 37° C. Following incubation with the detection antibody, the microtiter plates were washed and incubated at 37° C. for 30 minutes with a TMB (3,3′,5,5′-tetramethyl Benzidine) peroxidase substrate. The reaction was stopped by the addition of 2N sulfuric acid (H₂SO₄), and the absorbance of each well was read at 450 nm with 655 nm reference (A₄₅₀ₙₘ-A₆₅₅ₙₘ).

TABLE 1
Idursulfase Calibration Curve Standard Dilutions

| | | Final Idursulfase |
|---|---|---|
| | Dilution | Standard Concentration |
| Idursulfase Solution (μL) | Buffer (μL) | (ng/mL) |
| 10 μL of 2 μg/mL idursulfase | 990 | 20.0 |
| 500 μL of 20 nglmL idursulfase | 500 | 10.0 |
| 500 μL of 10 ng/mL idursulfase | 500 | 5.0 |
| 500 μL of 5 ng/mL idursulfase | 500 | 2.5 |
| 500 μL of 2.5 ng/mL idursulfase | 500 | 1.25 |
| 500 μL of 1.3 ng/mL idursulfase | 500 | 0.625 |
| — | 500 | 0.0 |

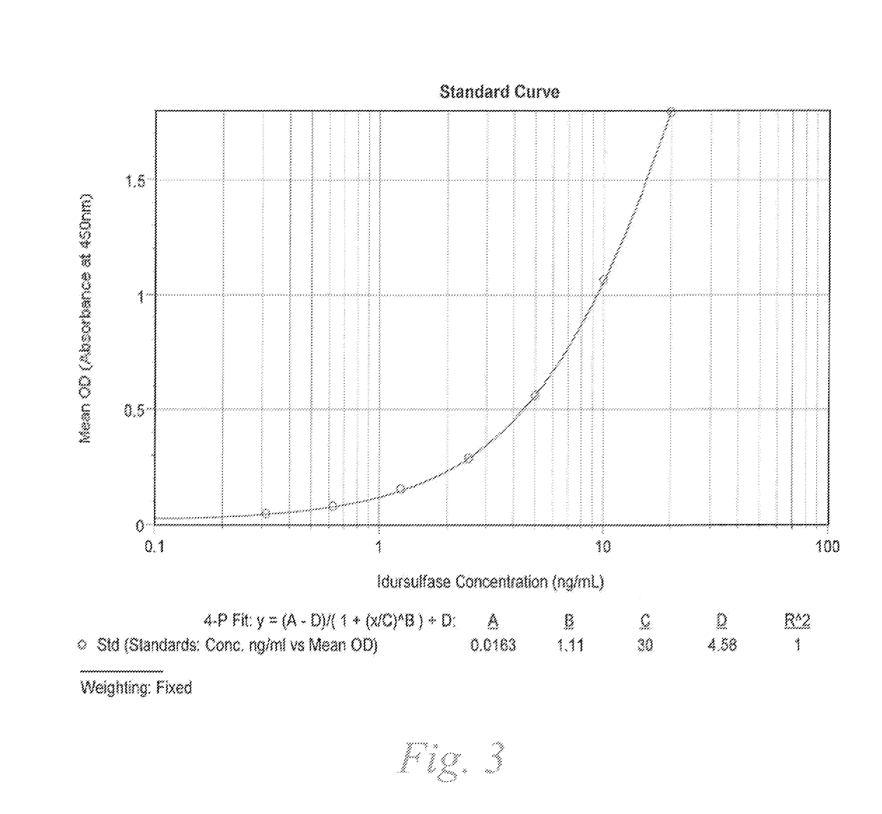

The concentration of idursulfase in the pre-incubated samples and controls was then calculated from the absorbance values based on the idursulfase standard curve (0-20 ng/mL) prepared based upon measurements of the standards obtained in the same microtiter plate. The standard curve was fit by a 4-parameter model, and the mean idursulfase result for each sample was recorded and is illustrated in FIG. 3 and in Table 2 below.

TABLE 2
Idursulfase Standards (ng/mL)

| Sam- | Conc. | | | Mean | | Mean | | CV |
|---|---|---|---|---|---|---|---|---|
| ple | ng/ml | Wells | BackCal | ng/ml | OD | OD | SD | % |
| Std1 | 20.00 | A1 | 20.06 | 19.99 | 1.796 | 1.792 | 0.006 | 0.3 |
| | | A2 | 19.93 | | 1.788 | | | |
| Std2 | 10.00 | B1 | 10.04 | 10.03 | 1.062 | 1.061 | 0.001 | 0.1 |
| | | B2 | 10.03 | | 1.060 | | | |
| Std3 | 5.00 | C1 | 4.91 | 4.95 | 0.558 | 0.562 | 0.005 | 1.0 |
| | | C2 | 4.99 | | 0.565 | | | |
| Std4 | 2.50 | D1 | 2.51 | 2.51 | 0.291 | 0.291 | 0.000 | 0.1 |
| | | D2 | 2.51 | | 0.291 | | | |
| Std5 | 1.25 | E1 | 1.28 | 1.28 | 0.150 | 0.151 | 0.001 | 0.6 |
| | | E2 | 1.29 | | 0.151 | | | |
| Std6 | 0.63 | F1 | 0.63 | 0.63 | 0.078 | 0.079 | 0.001 | 1.0 |
| | | F2 | 0.64 | | 0.080 | | | |
| Std7 | 0.31 | G1 | 0.28 | 0.30 | 0.042 | 0.044 | 0.002 | 4.5 |
| | | G2 | 0.31 | | 0.045 | | | |
| Std8 | 0.00 | H1 | Range? | 0.00 | 0.014 | 0.015 | 0.002 | 13.1 |
| | | H2 | 0.00 | | 0.017 | | | |

Smallest standard value: 0.015
Largest standard value: 1.792

The presence of conformation specific antibodies in patient serum samples allows the formation of antigen-antibody complexes during the sample pre-incubation step, resulting in a corresponding decrease in the amount of idursulfase detectable by this assay. Results obtained from patient serum samples following idursulfase enzyme replacement therapy at various time points were then compared to patient baseline results obtained from the same patient's serum sample prior to therapy (time zero). The conformation specific antibody ratio (Resultₚₒₛₜ₋ₜᵣₑₐₜₘₑₙₜ/Result_{baseline}) may then be calculated and reported.

### CSA Validation

For assay validation purposes, results obtained from positive control serum samples were compared to results obtained from negative control serum samples. The conformation specific antibody ratio (CSA ratio) was calculated as Result_{positive serum}/Result_{negative serum}. The assay acceptance criteria parameters and validation results obtained are summarized in Table 3 below, and the corresponding validation studies for determining the precision, linearity and sensitivity of the assay are further described below.

TABLE 3
Validation Protocol Acceptance Criteria and Validation Results

| Validation | Acceptance | |
|---|---|---|
| Parameter | Criteria | Validation Result |
| Assay Positive Cut | Mean CSA | ≦0.76 |
| Point | ratio - | |
| | 1.645 SD | |
| Precision | RSD ≦ 15% | RSD_{repeatability} = 0.6-7.8% |
| Repeatability & | | RSD_{reproducibility} = 4.3-10.6% |
| Reproducibility | | |
| Ruggedness | RSD ≦ 15% | RSD_{inter-analyst} = 2.0-9.6% |
| Inter-Analyst, | | RSD_{inter-day} = 1.9-9.5% |
| Inter-Day, and | | RSDₒᵥₑᵣₐₗₗ = 4.3-8.5% |
| Overall Precision | | |
| Robustness | RSD ≦ 15% | RSD_{sample preparation} = 1.8-6.9% |
| | | RSD_{pre-incubation} = 1.1-7.1% |
| | | RSD_{incubator} = 5.2-8.7% |
| Range of | R² ≧ 0.980 | R² = 0.991 (0-20 ng/mL) |
| Linearity | | |
| Sensitivity: | Based on study | LOD = 1.25 ng/mL |
| LOD and LOQ | results | |
| Stability | Recovery ≧ | % Recovery_{freeze-thaw} = |
| | 85.0% | 102.8-107.2% |
| | baseline | % Recovery_{Idursulfase buffer 4° C.} = |
| | | 93.3-107.9% |

### Assay Precision

To determine the precision of the assay, five independent assays were conducted by a single operator over a five day period. On each day, a negative control serum and a positive control serum were thawed and diluted in dilution buffer either with or without idursulfase as illustrated in Table 4 below to generate high, mid or low levels of positive control (PC) samples. The final serum concentration in each PC sample was 1:50 titrated with negative control (NC) serum. Three replicates of each sample were tested in duplicate.

TABLE 4
Sample Preparation

| | | | | 1X Idursulfase |
|---|---|---|---|---|
| Sample ID | Negative Control | Positive Control | Dilution Buffer | Dilution Buffer |
| 8 ng/mL I2S | — | — | — | 400 μL |
| NC 1:50 in 0 ng/mL | 8 μL NC Serum | — | 392 μL | — |
| NC 1:50 in 8 ng/mL | 8 μL NC Serum | — | — | 392 μL |
| NC 1:100 in 8 ng/mL | 4 μL NC Serum | — | — | 396 μL |
| PC 1:100 in 0 ng/mL | 4 μL NC Serum | 4 μL PC Serum | 392 μL | — |
| PC 1:100 in 8 ng/mL | 5 μL NC Serum | 5 μL PC Serum | — | 490 μL |
| PC 1:500 in 8 ng/mL | 6.4 μL NC Serum | 80 μL PC 1:100 | — | 314 μL |
| PC 1:1000 in 8 ng/mL | 7.2 μL NC Serum | 40 μL PC 1:100 | — | 353 μL |

The concentration of idursulfase in the pre-incubated samples was calculated from the absorbance using an idursulfase standard curve measured in the same ELISA plate. The CSA ratio was then calculated using the equation Result_{Positive Serum}/Result_{Negative serum}; wherein Result_{Negative serum} corresponds to the results of the negative control (NC) 1:50 in 1× idursulfase dilution buffer and Result_{positive serum} corresponds to the results of the positive control (PC) 1:100, 1:500 or 1:1000 in 1× idursulfase dilution buffer.

To determine the intra-assay repeatability, the mean, standard deviation (SD) and percent relative standard deviation (% RSD) were calculated from three results per sample per day. The % RSD was used to determine intra-assay precision. To determine the inter-assay reproducibility, the mean, SD and % RSD were calculated from fifteen results per sample obtained over five days. The % RSD was used to determine inter-assay precision. As illustrated in Table 5 below, results obtained from the precision study were all within the acceptance criteria validation protocol (RSD≦15%), therefore confirming the precision of the instant assay.

TABLE 5
Precision (CSA Ratio)

| | Sample ID | | | | | |
|---|---|---|---|---|---|---|
| | PC | PC | PC | PC | PC | PC |
| | 1:100 | 1:100 | 1:500 | 1:500 | 1:1000 | 1:1000 |
| Assay Reference | Mean | % RSD | Mean | % RSD | Mean | % RSD |
| Intra - Day 1 | 0.13 | 3.4% | 0.46 | 2.1% | 0.67 | 2.2% |
| Intra - Day 2 | 0.11 | 3.2% | 0.43 | 0.6% | 0.66 | 0.8% |
| Intra - Day 3 | 0.12 | 7.8% | 0.45 | 4.9% | 0.65 | 3.7% |
| Intra - Day 4 | 0.11 | 2.2% | 0.43 | 0.8% | 0.67 | 1.9% |
| Intra - Day 5 | 0.10 | 2.5% | 0.39 | 1.2% | 0.60 | 2.3% |
| Inter Assay | 0.11 | 10.6% | 0.43 | 5.8% | 0.65 | 4.3% |
| (five days) | | | | | | |

### Assay Linearity/Sensitivity

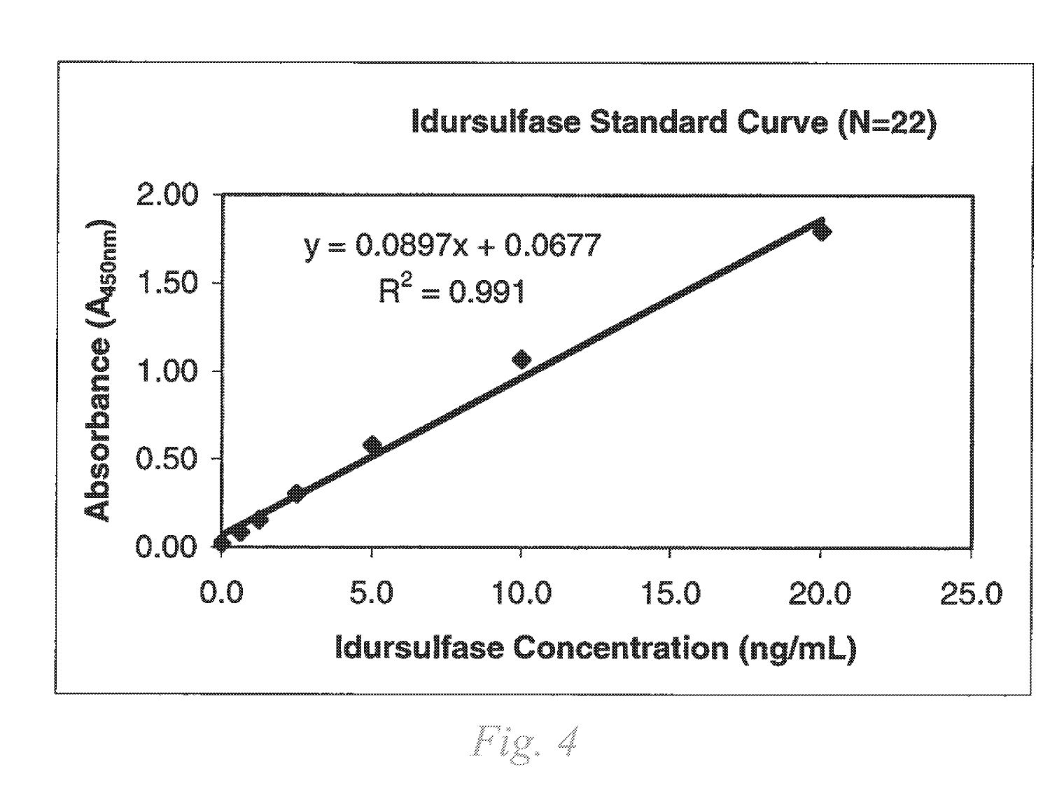

The linearity range of the assay was determined using 22 idursulfase standard curves (0-20 ng/mL) ran in the validation study assays. The mean, SD, and % RSD were calculated from the mean absorbance value of the 22 idursulfase standard curves. As illustrated in Table 6 below, the R² value for the mean idursulfase standard curves (0-20 ng/mL) was 0.991 and within the acceptance criteria set in the validation protocol (R²≧0.980). The R² values for each of the 22 individual standard curves were all within the acceptance criteria set in the validation protocol. The most linear section of the standard curve was between 0-10 ng/mL idursulfase, as demonstrated by the 0.999 R² shown in Table 6 and depicted in FIG. 4. The LOD value for this assay is defined as the lowest calibrator concentration which passes system suitability, 1.25 ng/mL (Table 6). The foregoing therefore confirms the linearity and sensitivity of the present assay.

TABLE 6
Assay Linearity

| Standard (ng/mL) | Mean | % RSD |
|---|---|---|
| 20.00 | 1.796 | 12.3% |
| 10.00 | 1.066 | 12.1% |
| 5.00 | 0.578 | 12.8% |
| 2.50 | 0.303 | 12.8% |
| 1.25 | 0.156 | 13.4% |
| 0.625 | 0.085 | 18.0% |
| 0.00 | 0.021 | 23.6% |
| R² (0-10 ng/mL) | 0.999 | |
| R² (0-20 ng/mL) | 0.991 | |

The results for the foregoing validation studies met all acceptance criteria set in the validation protocol (Table 3), therefore validating the assay for its intended use.

### Example 2

The present studies further describe the assay that was used to screen for conformation specific antibodies against idursulfase in serum samples obtained from some of the patients participating in the Hunter Outcome Survey (HOS).

### Material and Methods

As previously discussed, the HOS database, initiated in 2005, is overseen by national, regional, and global scientific advisory boards led by physicians who are experienced in the management of Hunter syndrome. Part of the role of the advisory boards is to supervise the analysis of data collected from national, regional, and global cohorts of patients.

Data entry and analysis in HOS were conducted as described previously. (See, Wrait, et al., Genet Med. 10 (2008) 508-516). A computer-based application is used to collect data and connects via the Internet to the database server using the Secure Socket Layer protocol. Data can therefore be entered remotely, at hospital/physician centers, through secured connections. The processing of data in the HOS database has been adapted to comply with the Swedish Personal Data Act (1998:204) and the EU Directive 2002/58/EC (Jul. 12, 2002) on the processing of personal data and the protection of privacy in the electronic communication sector. All US-based centers and laboratories or entities providing support for this survey, must, where applicable, comply with the Health Insurance Portability and Accountability Act of 1996 (HIPAA). Independent Review Board/Ethics Committee approval was obtained in the standard manner and according to local regulations in all participating centers and countries. All enrolled patients, their parents or a legal representative, have provided written informed consent for participation in HOS.

To investigate the development of IRR events relative to the initiation of treatment with idursulfase, the present analysis was limited to patients who started enzyme replacement therapy (ERT) at or after enrollment in HOS. Information on adverse events was collected in HOS, including description of the event, its timing, and any possible or probable relationship with the administered idursulfase agent (judged by the attending physician) was entered into the HOS database by the clinic. Events were coded as either infusion-related or not infusion-related. Infusion related reactions (IRR) were defined as events occurring during or within 24 hours of an infusion and with evidence of a causal relationship with administered idursulfase agent.

Anti-idursulfase antibody determinations were performed at Shire HGT Bioanalytics Department. All assays were developed and validated according to international guidelines (US FDA Center for Drug Evaluation and Research. “Guidance for Industry: Bioanalytical Methods Validation”. May 2001; ICH, Q2(R1): “Validation of Analytical Procedures: Text and Methodology’ November 2005; EMEA. “ICH Topic Q2B: Validation of Analytical Procedures: Methodology (CPMP/ICH/281/95), ICH Topic Q2B, Step 4 Consensus Guideline” November 1996) and antibody positive cut-off points were validated as recommended in Mire-Sluis, et al. (Mire-Sluis, et al., J Immunol Methods. 289 (2004) 1-16.)

Serum samples were screened using the conformation-specific anti-idursulfase antibody (CSA) assay described in Example 1 above. As previously discussed, conformation specific antibodies in serum samples form antigen-antibody complexes during the sample pre-incubation step, resulting in a corresponding decrease in the amount of free idursulfase detectable based on the formation of such antigen-antibody complexes in solution and measurement of such complexes by competitive immunometric enzyme-linked immunosorbent assay (ELISA). Briefly, serum samples were pre-incubated at room temperature for 2 hours with idursulfase to allow the formation of antigen (idursulfase)-antibody complexes. The pre-incubated samples were then incubated at 37° C. for 1 hour on microtiter plates previously coated with polyclonal goat anti-idursulfase antibody. The plates were incubated with a horseradish peroxidase (HRP)-conjugated goat anti-idursulfase antibody for 1 hour at 37° C. and incubated for a further 30 minutes with the peroxidase substrate 3,3′,5,5′-tetramethyl benzidine (TMB). The reaction was stopped using 2N sulfuric acid and the absorbance at 450 nm was measured. The plates were washed between all incubations. The concentration of idursulfase in each sample was calculated based on a concurrent idursulfase calibration curve. Results were reported as the ratio of the result from a patient test sample to that of the patient baseline sample. A ratio of 0.76 was validated as the cut-off point for detection of anti-idursulfase antibodies. A confirmatory assay was performed for test samples for which this ratio was ≦0.76.

In addition, all samples were screened for anti-idursulfase IgE, and any sample that tested negative by CSA was also tested for IgG antibodies using indirect ELISA methods. Briefly, microtiter plates were coated with idursulfase, washed, blocked, and then incubated with the serum samples. This was followed by incubation with HRP-conjugated secondary antibody (goat anti-human IgG or IgE), followed by TMB. The plates were washed between all incubations. The reaction was stopped and the absorbance measured as above. Validated cut-off points were absorbance of 0.057 for IgG and 0.031 for IgE. Confirmatory assays were performed on samples with absorbance greater than or equal to these values.

The presence of IgG antibodies was confirmed by radioimmunoprecipitation (RIP) assay. In this assay, patient sera were incubated for 2 hours at room temperature with ¹²⁵I-idursulfase, forming antigen/antibody complexes that were recovered using Protein G microbeads and quantified using a gamma counter. The radioactive count measured was proportional to the concentration of anti-idursulfase IgG antibodies in the test sample. Samples were confirmed positive when the validated ratio of the test sample counts to the positive control counts was >0.180. Samples that were negative by the RIP assay were reported as negative; IgG or IgE titer was measured in the positive samples and reported. All confirmed positive samples were additionally tested for neutralizing activity by an in vitro enzyme inhibition assay.

The in vitro enzyme inhibition assay measures neutralizing antibodies that interfere with the idursulfase catalyzed hydrolysis of the substrate 4-methylumbelliferyl sulfate (4 MUS). Idursulfase was mixed 1:1 with sample and incubated for 1 hour at 37° C. Next, the mixture was diluted in acetate buffer pH 4.5 and immediately added to 4-MUS in a 96-well microtiter plate in order to measure residual enzymatic activity. The reaction was stopped with a carbonate-glycine buffer pH 10.7 after 1 hour incubation at 37° C. and the fluorescence was measured (excitation, 360 nm; emission 465 nm). A concurrent 4 MUS calibration curve was performed. Each patient sample was tested alongside a sample obtained at baseline, and the result was reported as percent inhibition relative to the baseline sample. Samples that inhibited idursulfase activity by more than the validated cut-off point of 40% were considered to be positive for neutralizing antibodies.

### Results—Antibody Formation

Clinics participating in HOS typically provide samples for antibody determination only if an IRR event has occurred; in such instances, serum samples are taken no later than 1 day after the IRR event. However, because some clinics provide samples on a routine basis, not all specimens are associated with an IRR event. Serum antibody levels were measured for 63 patients in the study population; 32 (50.8%) tested positive for IgG antibodies to idursulfase at some point during the first year of ERT. Antibodies were considered to have neutralizing activity in 10 of these patients, although not necessarily in samples submitted at the time of an IRR event. In 4 of these 10 patients, an IRR event that was associated with a positive IgG result (not necessarily neutralizing) was reported in HOS. IgE antibodies towards idursulfase were not detected in any of the patients in this analysis.

For 10 of the IRR events reported in the current study, there was information on the presence of IgG antibodies to idursulfase from serum samples drawn within 1 day of the event. The 10 IRR events occurred in 7 of the 33 patients. Four of these events for which serum samples were taken were experienced by a single patient. As two of these IRR events occurred within a 24-hour period, analysis of antibodies related to these two events was therefore performed using a single serum sample. Of the 9 samples collected, 7 (from 6 patients) were positive for IgG to idursulfase. Two of these IgG-positive samples were from the patient mentioned above. Antibody neutralizing activity was tested in all seven of the IgG-positive samples. Neutralizing activity was found in two of the seven samples. Five samples (from 5 different patients) were not considered to have neutralizing activity in vitro.

The foregoing therefore further supports the ability of the conformation-specific anti-idursulfase antibody assay and related methods described herein to accurately detect the presence of interfering antibodies in biological samples. The assays, methods and kits described herein therefore provide a useful tool that may be used to further monitor patients receiving ERT and in certain embodiments may serve to predict the incidence or development of IRRs in such patients. Such assays, methods, kits and the information provided therefrom may serve to identify patients that require additional monitoring during ERT or to identify patients that may be candidates for home infusion therapy.

What is claimed is:

1. A method of determining the presence of a target antibody in a patient biological sample which binds an agent administered to the patient comprising:
(a) obtaining a biological sample from a patient to whom an agent of interest has previously been administered;
(b) contacting the biological sample with a predetermined amount of the agent of interest under conditions appropriate for binding of the agent to a target antibody in the biological sample, wherein if the target antibody is present an agent-target antibody pair is produced;
(c) subsequently contacting the biological sample with a capture agent capable of binding to the agent of interest to produce a captured agent of interest;
(d) removing unbound biological sample components;
(e) contacting the captured agent of interest with a signaling agent capable of binding to the captured agent of interest;
(f) removing unbound signaling agent; and
(g) comparing the presence of bound signaling agent to a control,
thereby determining the presence or absence of the target antibody in the biological sample.

2. A method according to claim 1 wherein the capture agent is immobilized.

3. A method according to claim 1 wherein the control comprises captured agent of interest that was not previously exposed to the target antibody.

4. A method according to claim 1 further comprising in step (g) comparing the amount of bound signaling agent to a control, thereby determining the amount of target antibody in the biological sample.

5. A method according to claim 1 wherein the agent of interest is a therapeutic agent.

6. A method according to claim 1 wherein the agent of interest is an enzyme.

7. A method according to claim 1 wherein the agent of interest is idursulfase.

8. A method according to claim 1 wherein the plurality of immobilized capture agents are immobilized on a solid planar surface.

9. A method according to claim 1 wherein the plurality of immobilized capture agents are a plurality of immobilized antibodies.

10. A method according to claim 1 wherein removing unbound biological sample components comprises a washing step.

11. A method according to claim 1 wherein the signaling agent is a labeled agent.

12. A method according to claim 1 wherein the capture agent is a competitive inhibitor of the target antibody for binding to the agent of interest.

13. A method according to claim 1 wherein the capture agent binds to the same physical epitope on the agent of interest as is bound by the target antibody.

14. A method according to claim 1 wherein the capture agent binds to the same conformational epitope on the agent of interest as is bound by the target antibody.

15. A method according to claim 1 wherein the signaling agent is labeled with a light-emitting label.

16. A method according to claim 1 wherein the signaling agent is labeled with HRP.

17. A method according to claim 1 wherein the signaling agent is a labeled antibody.

18. A method according to claim 1 wherein removing unbound signaling agent comprises a washing step.

19. A method according to claim 1 wherein the control is a standard derived from the amount of bound signaling agent in the absence of target antibody in the biological sample.

20. A method according to claim 1 wherein the control is a standard curve plotting amount of bound signaling agent in the presence of varying amounts of target antibody in the biological sample.

21. A method according to claim 1 wherein if the presence of target antibody is detected in the biological sample, the method comprises a further step of conducting a confirmatory assay.

22. A method according to claim 1 wherein if the presence of target antibody is detected in the biological sample, target antibody is further tested for neutralizing properties with regard to the agent of interest.

23. A method according to claim 22 wherein said target antibody is isolated from the biological sample prior to being tested for neutralizing properties.

24. A method according to claim 1 wherein the biological sample comprises serum.

25. A method according to claim 1 wherein the biological sample comprises cerebrospinal fluid (CSF).
